(19) United States
(12) Patent Application Publication
(10) Pub. No.: US 2020/0093866 A1
(43) Pub. Date: Mar. 26, 2020

(54) Use of selected single cobrotoxin molecule as an analgesic

(71) Applicant: zhankai Qi, Richmond (CA)

(72) Inventor: zhankai Qi, Richmond (CA)

(21) Appl. No.: 16/403,651

(22) Filed: May 6, 2019

(30) Foreign Application Priority Data
Sep. 25, 2018 (CN) 201811112055.6

(51) Int. Cl. A61K 35/583; A61K 38/17; A61K 9/00; A61P 25/04

(52) U.S. Cl. CPC A61K 35/583; A61K 38/1703; A61P 25/04; A61K 9/0043; A61K 9/0019

(57) ABSTRACT

A composition of matter for an analgesia and its method of use is disclosed. The composition comprises selected single cobrotoxin molecule which is characterized by its high affinity binding to nicotinic acetylcholine receptors, rapid onset, and better safety profile comparing to a cobrotoxins complexes. The method of use is for the treatment of pain, especially for the treatment of refractory pain as associated with advanced cancer, rheumatoid arthritis, chronic neuropathic, migraine, and viral infections.

### BACKGROUND OF THE INVENTION

### 1. Field of the Invention

The present invention relates to a composition of matter of selected single cobrotoxin molecule and its method for the treatment of chronic and acute pain, especially to the treatment of refractory pain as associated with advanced cancer. The pain associated with rheumatoid arthritis, chronic neuropathic, chronic headache, migraine, herniated intervertebral disc, lumbago, posttraumatic, burn wounds and viral infections could also be treated with the present invention. The composition consists of Chain A, Cobrotoxin, or Chain A, Cobrotoxin B; and acceptable carrier base for either parenteral, oral, sublingual, nasal, rectal or topical administration.

### 2. Description of the Prior Art

Relations between the structures and functions of the snake venom toxins were actively being investigated as therapeutic targets for the treatment of different conditions. Studies over the last few decades found that snake venom toxins belong to a small number of superfamilies of enzymes and non-enzymatic proteins. Some of the well-recognized families of venom proteins are: (i) three-finger toxin family (3FTxs); (ii) proteinase inhibitor family; (iii) lectin family; (iv) phospholipase A2 (PLA2) family; (v) serine proteinase family; and (vi) metalloproteinase family. The proteins within each family share remarkable similarities in their primary, secondary and tertiary structures, but they may differ from each other in their pharmacological effects. Thus, structure-function relationships and the mechanisms of action of snake venom proteins are intriguing and pose exciting challenges (R. Manjunatha Kini et al., 2010).

The family of non-enzymatic polypeptides contains 60-74 amino acid residues (Dufton and Hider, 1988; Endo and Tamiya, 1991) and they belong to the three finger toxin family (3FTxs) which are found in the venoms of elapids (cobras, kraits and mambas), hydrophiids (sea snakes), and colubrids (Pawlak et al., 2006, 2009). Furthermore, they are classified as short-chain neurotoxins (60-62 amino acid residues and four disulfides), long-chain neurotoxins (66-74 amino acid residues and five disulfides) (Endo and Tamiya, 1991), and non-conventional 3FTxs neurotoxins (R. Manjunatha Kini et al., 2010).

Neurotoxins were first isolated from snake venom approximately 50 years ago and were found bonded to acetylcholine receptor and nicotinic acetylcholine receptor. Scientists (Chang and Lee, 1963) utilized zone electrophoresis on starch to isolate a-bungarotoxin, b-bungarotoxin, and g-bungarotoxin from the venom of the many-banded krait (Bungarus multicinctus), these neurotoxins were subsequently examined for pharmacological activity in a range of in vitro preparations and in mice to determine lethality (i.e. LD50 values). Using the chick biventer cervicis nerve-muscle preparation (Chang and Lee, 1963) showed that a-bungarotoxin inhibited nerve-mediated (i.e. indirect) twitches and abolished contractile responses to acetylcholine, indicative of a postsynaptic mode of action. The discovery of a-bungarotoxin lead to the characterization of the nicotinic acetylcholine receptor (Changeux et al., 1970), which is now one of the most well characterised ionotropic receptors (Nirthanan et al., 2004).

Nicotinic acetylcholine receptors (nAChRs) bind to acetylcholine and transmit its signal, they play an important role in neurotransmission including neuropathic and inflammatory pain (Arik J. Hone et al., 2017; Deniz Bagdasab et al., 2015).

The group of snake neurotoxins that have postsynaptic blocking actions are now widely referred to as a-neurotoxins, they interfere with cholinergic transmission at various post-synaptic sites by binding to nAChRs in the peripheral and central nervous systems. (Changeux, 1981; Ruan et al., 1990, 1991; Carmel M et al., 2013; V. I. Tsetlin et al., 1982; Abraham O. Samson, TaliScherf, 2002; V.I Tsetlin aFHuchob, 2004).

Neurotoxins from elapid species have well-established acetylcholine receptors and modes of action at the molecular level (Changeux, 1981; Ruan et al., 1990, 1991). The Study of “Inhibition of the Nicotinic Acetylcholine Receptors by Cobra Venom α-Neurotoxins” (Angela Alama et al., 2011) confirmed Inhibition of the Nicotinic Acetylcholine Receptors by Cobra Venom α-Neurotoxins; The Study in “Hunter's Tropical Medicine and Emerging Infectious Disease”(David A Warrell, 2013) demonstrated postsynaptic α-neurotoxins, such as α-bungarotoxin and cobrotoxin, binding to acetylcholine receptors on the motor end-plate, like curare, competitively inhibiting acetylcholine. Neurotoxin extracted from Cobra venom is referred to as a cobrotoxin, there are about 20 cobrotoxin single molecules that have been isolated from Naja Atra according to the NCBI protein data bank.

Central nervous system (CNS) is the major target site of cobrotoxins for pain control. In the study of “The effect of cholinergic manipulations on the analgesic response to cobrotoxin in mice”, Intracerebroventricular (i.c.v.) injection of cobrotoxin, a neurotoxin isolated from the venom of Naja naja atra, produced an antinociceptive response in mice as measured by the tail-flick test. This effect of cobrotoxin was blocked by systemic administration of atropine, but not by methylatropine or naloxone. Depletion of central acetylcholine (ACh) by hemicholinium-3 (HC-3) blocked the antinociceptive action of cobrotoxin. These results suggest that central cholinergic neurons are important for the mediation of the antinociceptive properties of cobrotoxin (Chen and Robinson, 1990). In another study “The effect of cobrotoxin in the turnover of acetylcholine in the mouse brain”, cholinergic neurons in the hypothalamus is selectively suppressed by cobrotoxin which confirm that the active site of cobrotoxin is at the central nervous system level (Chen and Robinson, 1990.02). Further studies, namely “The effect of cobrotoxin on cholinergic neurons in the mouse brain” show that by using multiple time-point constant-rate infusions of deuterium-labeled phosphorylcholine, appropriate kinetic parameters were obtained for use in the calculation of the turnover rate of acetylcholine (TRACh) in selected mouse brain regions. These results suggest that the activity of hippocampal and midbrain cholinergic neurons is suppressed by cobrotoxin (Chen and Robinson, 1992), which further confirmed cobrotoxin's target site at the CNS level.

A cobrotoxins complexes extracted from cobra venom was once approved as an analgesia because of its anti-nociceptive function and non-opioid system involvement. However, safety issues such as respiratory inhibition due to synergism of cobrotoxins complexes still pose as big concerns. Additionally, slow onset and inconstant clinical effects prevent further use of cobrotoxins complexes in clinics. The product was temporarily taken off the market. The regulatory body asked the manufacturers to come up with a GMP standard product of higher purity and, possibly, with higher bio-activity.

The cobrotoxins complexes product once on the Chinese market was known as a general neurotoxins extracted from crude venom, the labeling did not refer to a special type of cobrotoxin or cobrotoxin molecule. The quality control measures are neurotoxin proteins in the composition should be over 80%. Lack of information about the exact active ingredients and the remaining impurity caused safety concerns as the complexes of snake venom toxins can cause synergism which can lead to some severe adverse event such as respiratory inhibition.

As previously mentioned, snake venom contains different toxin families which include neurotoxins, cytotoxins, cardiotoxins, Phospholipase A2 (PLA2s), emotoxins, nerve growth actor, lectins, metalloproteinases, disintegrins, crotoxin, serinoproteases, hyaluronidase, cholinesterases (Vagish Kumar et al; 2015). Phospholipase A2 (PLA2s) was most toxic, followed by cardiotoxin, with cobrotoxin being the least toxic (C. Y. Lee et al., 1977).

Synergism is a significant phenomenon present in snake venoms that may be an evolving strategy to potentiate toxicities. A synergism exists between different toxins or toxin complexes in various snake venoms. The predominant toxins, such as PLA2s, three-finger toxins (3FTxs), metalloproteases and serine proteases play essential roles in synergistic processes (Shengwei Xiong et al., 2018).

Synergism between venom toxins exists for a range of snake species. Two or more venom components interact directly or indirectly to potentiate toxicity to levels above the sum of their individual toxicities. From a molecular perspective, synergism may exist in two general forms:
(1) Intermolecular synergism, when two or more toxins interact with two or more targets on one or more (related) biological pathways, causing synergistically increased toxicity.
(2) Supramolecular synergism, when two or more toxins interact with the same target in a synergistic manner or when two or more toxins associate and create a complexes with increased toxicity (Laustsen, A. H. 2016).

An example of intermolecular synergism is α-neurotoxins from elapid(s) combined with other toxins, causing a synergistic potent toxic effect leading to flaccid paralysis and respiratory failure in victims and prey (Laustsen et al., 2015). In a study about how Mamba snake (Cobra) venom caused high mortality, scientists revealed that potassium channel blocking activity was only one of the mechanisms. There are various other pathways involved. The combined toxicity induced at various organ levels by different dendrotoxins leading to all-around damage was a concern towards high mortality (Anil Kumar et al., 2018).

Supramolecular synergism may arise when toxins form complexes with synergistic effects via a mechanism in which several venom components combine to create a hyper-potentiated toxin. Cobra cytotoxins have been shown to enhance PLA2 activity through complexes formation and deformation of cell membranes, causing cellular lysis due to hydrolysis of phospholipids (Gasanov et al., 1997, Gasanov et al., 2014).

Study of snake venoms showed that separated venom fractions from the venom of the related Eastern green mamba (Cobra) were devoid of lethality after 48 hours when administered alone, whereas the whole venom was lethal within 10 minutes when administered at a similar dose (Strydom D J, and Botes D P, 1970).

Synergistic toxins are known to enhance the toxicity of certain toxins.

Individually, these proteins are less toxic, but they act as potent toxins when injected into mice in combination. These toxins show similarities with that of neurotoxin or cytotoxins in amino acid sequence and number of half cysteines (Joubert and Taljaard, 1979). Such synergistically acting toxins are called synergistic toxins.

Neurotoxins act at the neuromuscular junction. They produce a flaccid paralysis of the voluntary muscles and cause death from respiratory obstruction and/or respiratory insufficiency (Campbell C H, 1975). Snake neurotoxins are the main toxic proteins of cobra, krait, tiger snake and sea snake venoms which block neuromuscular transmission and cause animals to die of respiratory paralysis (C. C. Yang, Natural Toxins 2). Respiratory distress (Angela Alama and Cristina Bruzzo, 2011), respiratory paralysis (C. C. Yang, Natural Toxins 2) or respiratory failure are the results of an animal bite (Pravat Thatoi et al, 2016), but it could also be the consequence of administering cobrotoxins complexes as indicated in the insert of the product.

Slow onset and inconstant clinical effect were another two issues that made the cobrotoxins complexes out of favor of medical practitioners. After administering cobrotoxins complexes as analgesic, slow onset after approximately 2-3 hours and inconstant analgesic effect were commonly reported, thus not meeting clinical requirements. (Liqi et al., 2003; Chen Ruzhu et al., 1988; Chen Yan et al., 2007; Zhu Tianxin et al., 2007).

Cobrotoxin proteins with molecular sizes ranging from approximately 6.5 KDa to 15 KDa could produce analgesic effects (Ming zhixue et al., 2013; Gao Hongjing, 2014). SDS-PAGE analysis of these proteins was shown in FIG. 1. Cobrotoxins are hydrophilic and the main mechanism to pass the blood-brain barrier (BBB) is passive transport. A cobrotoxin molecule of 15 KDa will need more time to cross the BBB than a molecule of 6.5 KDa because the increased molecular weight reduces BBB permeation (Edward H. Kerns, 2012).

According to pharmacokinetic researches, target exposure and target engagement have been identified as two out of three “pillars of success” of drug discovery programs (Morgan P et al., 2012). Prerequisites for drug target engagement is binding of a drug to its molecular target protein and the exposure of the target at concentrations in excess of the pharmacological potency of the compound for a sufficient period of time.

For a cobrotoxins complexes to reach an adequate concentration to satisfy drug target engagement, the overall time is highly dependant on the proportion of high molecular weight cobrotoxins and their abilities to cross BBB.

Pertaining to the inconsistency of clinical effect, the differences in each individual cobrotoxin molecule's affinity to bind with nAChR and the different composition of individual cobrotoxin molecule in each batch due to lack of precise quality control standard causes variations in the drug target engagement. Finally, the denaturation of proteins happens during the extraction process due to hydrolysis, temperature, and chemicals, but the current quality control procedure can't detect that. Overall, the aforementioned factors can affect the time of onset and constant clinical effects.

### SUMMARY OF THE INVENTION

The primary purpose of the invention is to provide a composition of matter using selected single cobrotoxin molecule for the treatment of pain with a high safety profile and a low risk of respiratory inhibition by eliminating all complexes cobra venom, save the selected cobrotoxin molecule from said composition.

The further purpose of the invention is to provide a composition of matter for the treatment of pain with rapid onset, which is about ⅓ of the onset time of cobrotoxins complexes based on pain responses in mice/rats via muscle or intravenous injection due to an superior ability crossing the BBB of selected cobrotoxin single molecule.

Finally, the invention is to provide a composition of matter that could meet patients' expectations with constant clinical effect in pain treatment due to the high affinity of selected cobrotoxin single molecule binding to nAChR.

### BRIEF DESCRIPTION OF THE DRAWINGS

FIG. 1 is a photograph of the separation of proteins from Naja Atra by electrophoresis using a discontinuous polyacrylamide gel as a support medium and sodium dodecyl sulfate (SDS) to denature the proteins.

FIG. 2 is the result of the isolation of Naja Atra venom by cation-exchange chromatography on an open column and reverse-phase HPLC; among a total of 12 fractions, fractions A and B were identified as high-affinity cobrotoxins molecules binding to nAChR in 125I labeled-αBtx-nAChR binding inhibition test.

FIG. 3 is an illustration of the quantitative micro-dialysis.

FIG. 4 the brain time-concentration curves of the selected single cobrotoxin molecules and a cobrotoxins complexes.

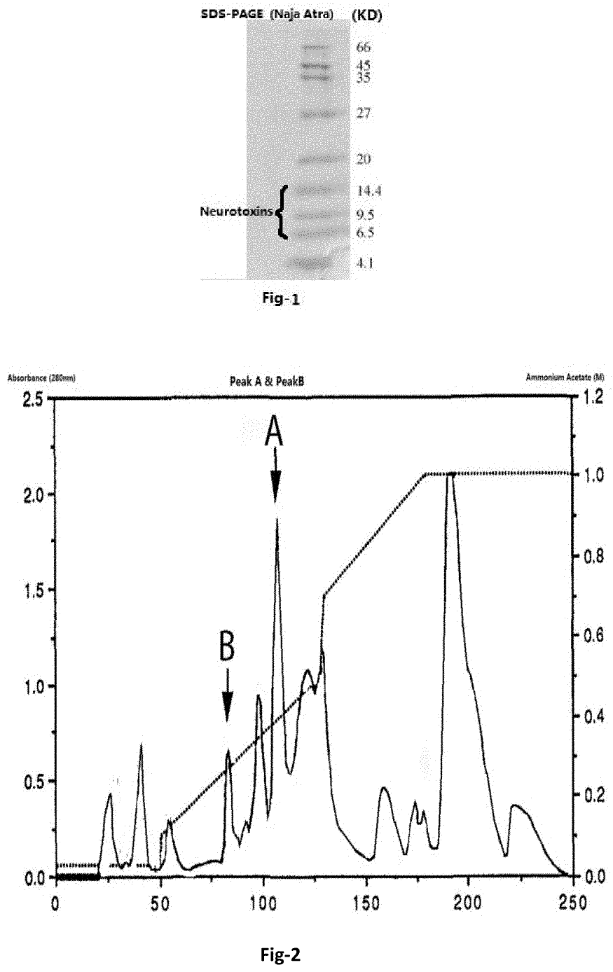

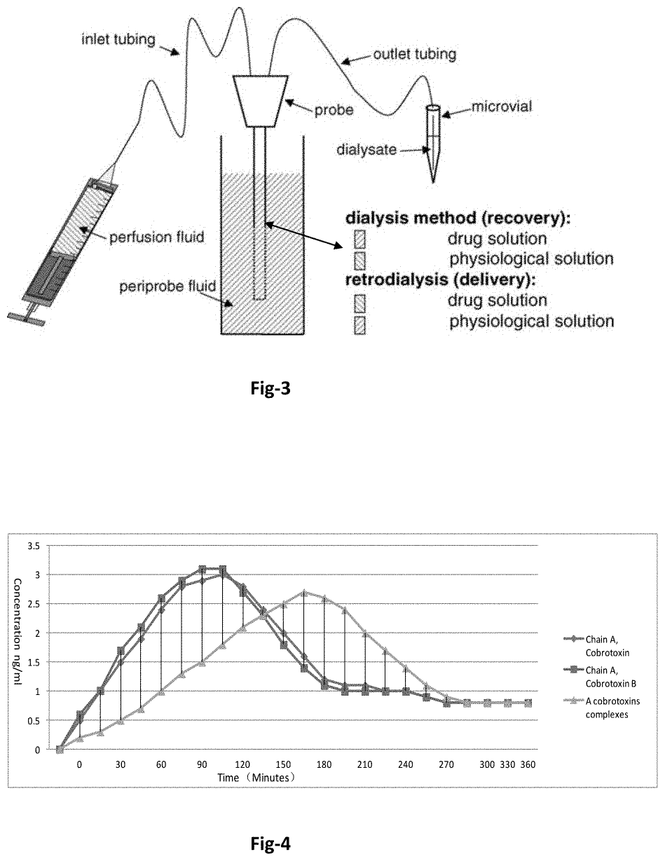

### DETAILED DESCRIPTION OF THE INVENTION

1) Separation and Purification of cobra venom toxins Based on lyophilized venom powder from Naja atra, a total of 12 fractions were isolated by cation-exchange chromatography on an open column (50×2.5 cm I.D.) packed with TSK CM-650(M). The process was performed and described in the following sequence:
i. Venom powder was dissolved in 10 ml of 0.025 M ammonium acetate (pH6.0).
ii. Starting buffer (20-50 mg/ml) was applied to TSK CM-650 column equilibrated with the same buffer.
iii. After the column had been washed with 300 ml of the initial buffer, the proteins adsorbed were eluted with a two-stage linear gradient (0.1-0.5 M and 0.7-1.0 M ammonium acetate buffer) as indicated in the FIG. 2.
iv. A reverse-phase HPLC (RP-HPLC) was performed on a Hitachi' liquid chromatograph with a model L-6200 pump. The column eluates (6 ml/tube/7.5 min) were monitored for absorbance at 280 nm.

As shown in FIG.-2, a total of 12 fractions from the aforementioned ion-exchange chromatography were further desalted and purified by a reverse-phase HPLC (RP-HPLC) with Vydac RP-C18 (4.6×250 mm, 5.0 um). Fractions A and B were identified as high-affinity cobrotoxins with enhanced binding ability to nAChR in a later 125I labeled-αBtx-nAChR binding inhibition test.
2) Screening and selection of single cobrotoxin molecule with high affinity binding to nAChR for better drug target engagement The 125I labeled-α-bungarotoxin(αBtx)-nAChR binding inhibition test was performed to evaluate the affinities of different cobrotoxins molecules. Both α-bungarotoxin and cobrotoxin bind to nAChR, and competitively inhibits counter part's binding ability. α-bungarotoxin is considered to have very high affinity with nAChR, it can competitively block nAChR at the acetylcholine binding sites in a relatively irreversible manner, so cobrotoxin's ability to inhibit α-bungarotoxin's binding to nAChR can represent cobrotoxin's affinity with nAChR. Higher the affinity of cobrotoxin with nAChR, greater the inhibitory rate toward α-bungarotoxin's binding to nAChR. In our radio-immunoprecipitation test, only nAChR bonded α-bungarotoxin will be precipitated with nicotinic acetylcholine antibody (mAb 35) and Rabbit Anti-Rat IgG, whilst unbounded α-bungarotoxin will be washed out. By testing the concentration of bonded α-bungarotoxin, we can determine the inhibitory rates of different cobrotoxins molecules isolated in step 1).

Calculation of “125I-αBtx-nAChR binding inhibitory rate (%)”

=100×(C_{BSA}−C_{cbx})÷(C_{BSA}−C_{αBtx}),

Where

C_{BSA} means concentration using beef serum albumin to inhibit 125I-αBtx-nAChR binding, 0% inhibited.

C_{αBtx} means concentration using α-bungarotoxin to inhibit 125I-αBtx-nAChR binding, 100% inhibited.

C_{cbx} means concentration using isolated cobrotoxin in step 1) to inhibit 125I-αBtx-nAChR binding.

The 125I-αBtx-nAChR binding inhibition experiment process is performed and described in following sequence:
i. A total of 0.5 ml nAChR crude extract from rat skeletal muscle was added to different purified cobrotoxin, then mixed with 1 ul mAb35 (5.9 mg/ml) and 1 ul 125I-αBtx (0.18 μg/ml), then blended at 4° C., and stored overnight for at least 10 hours.
ii. About 10 μl rabbit anti-rat IgG (4.5 mg/ml) was added to the aforementioned mixture on the next day and kept at 4° C. for two hours.
iii. The resultant from the previous steps was then centrifuged for five minutes at 13,000 rpm and the sediment was washed thrice with Triton X-100 lotion. The (Bq) value per second was measured using the gamma radioimmunoassay counter.
iv. Result:

Fractions A and B were in the range of approximately 40%-50% binding inhibition rates whilst other fractions were approximately between 0%-20% binding inhibition rates. Fractions A and B were purified and desalinated by RP-HPLC column (4.6×250 mm, VYDAC RP-C18) and were selected for further sequencing process.

3) Sequencing the primary structures of the selected cobrotoxin molecules (fractions A and B) to establish rigorous quality control standards to eliminate synergism and to improve consistency of target engagement Edman degradation method has been used to determine the amino acid sequence of fractions A and B, the process is performed and described in the following sequence:
i. A total of 5 nmol of proteins were dissolved in 100 μl of 0.1% trifluoroacetic acid (TFA).
ii. These samples were reduced with β-mercaptoethanol then reacted with iodoacetic acid.
iii. These reduced and carboxylmethylated (RCM)-proteins were treated with Glu-C and Arg-C endoproteinases then separated on RP-C18 HPLC.
iv. The resulting RCM-proteins were then subjected to automated Edman degradation to obtain the N-terminal sequences using a protein sequencer (Model 491A-ABI).
v. By comparing the amino acid compositions and primary sequences determined by the sequencer, the sequence assignment can be determined.

In our test, the amino acid sequence of fraction A protein was: lechnqqssq tptttgcsgg etncykkrwr dhrgyrterg cgcpsvkngi einccttdrc nn The name according to NCBI Data Bank is Chain A, Cobrotoxin.

The amino acid sequence of fraction B protein was: lechnqqssq tpttktcsge tncykkwwsd hrgtiiergc gcpkvkpgvn Inccttdrcn n The name according to NCBI Data Bank is Chain A, Cobrotoxin B.

4) Respiratory inhibition test Chain A, Cobrotoxin and Chain A, Cobrotoxin B were separately administered to 10 Kunming mice of 20±2 g. No respiratory inhibition was observed at effective dose of 60 μg/kg.

5) Comparison of the ability to cross the BBB and the time to reach effective brain concentration of Chain A, Cobrotoxin, Chain A, Cobrotoxin B with a cobrotoxins complexes

The estimated molecular weights of Chain A, Cobrotoxin, Chain A, Cobrotoxin B are 6.9±0.6 KDa; the estimated molecular weights of a cobrotoxins complexes extracted from crude venom ranged from approximately 6.5 KDa to 15 KDa. Quantitative microdialysis (QMD) is used to determine the absolute unbound brain extracellular fluid (bECF) concentration of Chain A, Cobrotoxin, Chain A, Cobrotoxin B, and the cobrotoxins complexes. QMD is the only technique that enables sampling of bECF in conscious animals and provides direct evidence of exposure at extracellular target sites (Kielbasa and Stratford, Jr. 2015)

QMD consists of implanting a probe in rat (FIG.-3). The membrane of the probe will allow the exchange of perfusate with bECF, so the concentration of Chain A, Cobrotoxin, Chain A, Cobrotoxin B, and a cobrotoxins complexes in bECF could be measured through dialysate. However the absolute drug concentration needs to be adjusted by taking into account the recovery rate of the probe as the recovery rate is less than 100% due to the fact that flow rate of perfusate through the probe does not allow sufficient time for the solute to equilibrate between the perfusate and bECF across the probe membrane (Li Di and Edward H. Kerns. 2015). In response, an in vivo recovery rate test of the probe (MD-2200) was performed on 125I-labeled Chain A, Cobrotoxin, Chain A, Cobrotoxin B, and a cobrotoxins complexes, each with three different concentrations of 5, 25, and 50 ng/ml as perfusate, with a flow rate of 2 μL/min was perfused through the probe. During microdialysis, the dialysate was collected every 10 minutes for an hour, then tested using SN629B-radioimmunoassay counter to calculate cpm. Finally, cpm was converted to cobrotoxin concentration. The recovery rate (by loss) % was calculated as:

=100=(C_{perfusate}−C_{dialysate})÷C_{perfusate},

where C_{perfusate} is the drug concentration in the perfusate, and _{Cdialysate} is the drug concentration in the dialysate. In our test, the average recovery rate (by loss) was 15%.

Microdialysis was performed to assess the free drug concentration of Chain A, Cobrotoxin, Chain A, Cobrotoxin B, and the cobrotoxins complexes in rats brain. A total of 15 rats of 320±20 g were categorized into three groups. After implantation of probes(MD-2200), they were perfused with artificial cerebrospinal fluid with a flow rate of 2 μL/min. Same dosage of 120 μg; 1.61×107 Bq/kg 125I-labeled Chain A, Cobrotoxin, Chain A, Cobrotoxin B, and the cobrotoxins complexes were administered respectively into the three groups by IV injection. The dialysate was collected on an interval of 10 minutes for 360 minutes, followed by the testing of the dialysate with SN629B-radioimmunoassay counter. Converting cpm to cobrotoxin concentration, adjusted by the recovery rate (by loss), the brain concentrations curves of Chain A, Cobrotoxin, Chain A, Cobrotoxin B, and the cobrotoxins complexes at different time points in bECF were obtained.

As shown in FIG.-4, during any time course, Chain A, Cobrotoxin and Chain A, Cobrotoxin B were always ahead of the cobrotoxins complexes in reaching a specific concentration. A composition of molecular size of 6.9±0.6 KDa had a faster ability to cross the BBB than the complexes of molecular weights ranging from 6.5 KDa to 15 KDa, and reached higher brain concentrations sooner. Another reason that might impact larger molecule cobrotoxins in reaching higher brain concentration is rapid renal clearance, because it takes the cobrotoxins 10 times longer to cross the BBB compared to the renal clearance speed. (Lin and Lu, 2009). Cobrotoxins of larger molecular weight has a higher chance to be excreted from circulating blood due to lower speed crossing the BBB.

Sufficient concentration is the “first pillar” for a drug to show its expected pharmacology activity (Morgan P et al., 2012). Insufficient brain exposure leaves many central nervous system (CNS) diseases untreated or without optimum drugs. In past years, a high percentage of promising CNS drug candidates have failed. A major cause of this failure is the restricted access of many drug candidates circulating in the blood to penetrate into the brain owing to the BBB (Li Di and Edward H. Kerns, 2015).

Cobrotoxins complexes, due to the difference in molecular size and in affinity binding to nAChR, cause not only delays in onset but also inconsistency in clinical effect. However, by identifying, isolating, and using the selected single cobrotoxin molecule with high-affinity binding to a target receptor and better ability to penetrate the blood-brain barrier, an new analgesic with a high safety profile, rapid onset, and constant clinical effect was formulated that could greatly improve the pharmacology activities.

### EXAMPLES

Effects of Chain A, Cobrotoxin and Chain A, Cobrotoxin B on Pain Responses in Mice/Rats

### Example 1

Tail flick test for evaluation of the analgesic effect of selected cobrotoxin molecule at CNS level

Most commonly, an intense light beam is focused on the animal's tail and a timer starts. When the animal flicks its tail, the timer stops and the recorded time (latency) is a measure of the pain threshold.

METHODS: Periaqueductal Gray (PAG) was injected with selected cobrotoxin molecule in rats, and the central analgesic effect was evaluated by the tail flick due to thermal radiation. 20 Wistar rats with a weight of 200±20 g were divided into 2 groups, 10 per group. Both Chain A, Cobrotoxin and Chain A, Cobrotoxin B, at 1.5, 3.0, and 6.0 μg/kg exhibited a dose-dependent increase in the latency induced by thermal radiation. The analgesic effect of Chain A, Cobrotoxin and Chain A, Cobrotoxin B appeared in 10˜15 minutes and peaked at 25˜30 minutes after drug administration. The maximum increase of pain threshold was over 200%.

The ED50 of the antinociceptive effect of the thermal radiation-induced tail flick for Chain A, Cobrotoxin and Chain A, Cobrotoxin B was 3.3˜4.0 μg/kg in the tail flick test.

### Example 2

Writhing test for the evaluation of analgesic effect by parenteral injection The writhing test is a chemical method used to induce pain of peripheral origin by injection of irritant principles like acetic acid in mice. Analgesic effect of the test compound is inferred from the decrease in the frequency of writhing.

METHOD: A total number of 60 Kunming mice were used, 10 per group, with a weight of 20±2 g. Chain A, Cobrotoxin and Chain A, Cobrotoxin B at 30, 45 or 60 μg/kg (iv) exhibited a dose-dependent decrease in the frequency of writhing induced by acetic acid. At dose of 60 μg/kg (iv), The analgesic effect started at 30 minutes, and could reach 60˜70% inhibitory rate of mice writhing at 90 minutes after administration. The ED50 of the anti-nociceptive effect of acetic acid induced writhing was 40˜50 μg/kg in the writhing test for Chain A, Cobrotoxin and Chain A, Cobrotoxin B.

### Example 3

Writhing test for the evaluation of analgesic effect by nasal administration METHOD: A total number of 60 Kunming mice were used, 10 per group, with a weight of 20±2 g. Nasal administration of Chain A, Cobrotoxin and Chain A, Cobrotoxin B at 30, 45 or 60 μg/kg exerted a dose-dependent decrease in the frequency of writhing induced by acetic acid. The analgesic effect started at 30 minutes and lasted at least for 6 hours after drug administration. ED50 of the anti-nociceptive effect of acetic acid induced writhing was 38˜46 μg/kg in the writhing test for Chain A, Cobrotoxin and Chain A, Cobrotoxin B.

### Example 4

Writhing test for the evaluation of analgesic effect by oral administration METHOD: A total number of 60 Kunming mice were used, 10 per group, with a weight of 20±2 g. Oral administration of Chain A, Cobrotoxin and Chain A, Cobrotoxin B at 160, 320 or 480 μg/kg exerted a dose-dependent decrease in the frequency of writhing induced by acetic acid. ED50 of the anti-nociceptive effect of acetic acid induced writhing was 300˜350 μg/kg in the writhing test for Chain A, Cobrotoxin and Chain A, Cobrotoxin B.

Although the invention herein has been described with reference to particular embodiments, it is to be understood that these embodiments are merely illustrative of the principles and applications of the present invention. It is, therefore, to be understood that numerous modifications may be made to the illustrative embodiments and that other arrangements may be devised without departing from the spirit and scope of the present invention as defined by the appended claims.

### REFERENCES

Abraham .Samson, TaliScherf. The Mechanism for Acetylcholine Receptor Inhibition by α-Neurotoxins and Species-Specific Resistance to α-Bungarotoxin Revealed by NM Neuron. Volume 35, Issue 2, 18 July, Pages 319-332. 2002

Alan J. Magill, MD, FACP, FIDSA, Edward T Ryan, David R Hill. Postsynaptic (α-)neurotoxins, such as α-bungarotoxin and cobrotoxin, are three-finger fold polypeptides that bind to acetylcholine receptors on the motor end-plate, like curare, competitively inhibiting acetylcholine. Hunter's Tropical Medicine and Emerging Infectious Disease Ninth Edition. 2012

Angela Alama, 1 Cristina Bruzzo. Inhibition of the Nicotinic Acetylcholine Receptors by Cobra Venom α-Neurotoxins: Is There a Perspective in Lung Cancer Treatment?, PLoS One, 6(6), 2011

Arik J. Hone, J. Michael McIntosh. Nicotinic acetylcholine receptors in neuropathic and inflammatory pain. FEBS PRESS.14 Oct., 2017

Anil Kumar, Varun Gupta. Neurological Implications of Dendrotoxin: A Review. EC PHARMACOLOGY AND TOXICOLOGY. May 25, 2018

Bouw M R. Mammarlund Udenaes M. Methodological aspect of the use of a calibrator in vivo microdialysis-further improvement of the retrodialysis method. Pharm Res 15:1673-1679.1998

Changeux, J. P. Harvey Lect. 75, 85-254. 1981

Carmel M. Barber a, Geoffrey K. alpha-neurotoxins have a similar biological property; namely, binding to acetylcholine receptors (AchR). Alpha neurotoxins. Toxicon (66) 47-58. 2013

Chang and Lee. Isolation of neurotoxins from the venom of Bungarus multicinctus and their modes of neuromuscular blocking action. Arch. Int. Pharmacodyn Ther. 144, 241-257. 1963.

Changeux, J.-P., Kasai, M., Lee, C.-Y. Use of a snake venom toxin to characterize the cholinergic receptor protein. Proc. Natl. Acad. Sci. USA 67 (3), 1241-1247. 1970

Chin-Chun Hung, Shih-Hsiung Wu. Two Novel a-Neurotoxins Isolated from Taiwan Cobra:Sequence Characterization and Phylogenetic Comparison of Homologous Neurotoxins. Journal of Protein Chemistry, Vol. 17, No. 2, 1998

Chen Ruzhu, Wu Xiurong. Analgesic effect of Cobrotoxin. Chinese Pharmacological Bulletin, 4(2): 113. 1988

Chen Yan, Xu Yunlu. Purification Characterization and analgesic activity of neurotoxin from Naja naja atra venon. Strait Pharmaceutical Journal, 19(12): 27, 2007

C. Y. Lee Y. M. Chen. Central neurotoxicity of cobra neurotoxin, cardiotoxin and phospholipase A2, Toxicon, Volume 15, Issue 5, Pages 395-401. 1977

Campbell C H. The effects of snake venoms and their neurotoxins on the nervous system of man and animals. Contemp Neurol Ser.;12:259-93, 1975

C. C. Yang, Natural Toxins 2, pp 85-96. Cite as Structure and Function of Cobra Neurotoxin, 1996

Daniel J. STRYDOM. Purification and Properties of Low-Molecular-Weight Polypeptides of Dendroaspis polylepis polylepis (Black Mamba) Venom. Snake Venom Toxins. Eur. J. Biochem. 69, 169-376. 1976

DenizBagdasab, Shakir D. AlSharariac. The role of alpha5 nicotinic acetylcholine receptors in mouse models of chronic inflammatory and neuropathic pain. Biochemical Pharmacology Volume 97, Issue 4, Pages 590-600. 15 Oct.,2015

Dufton and Hider. Structure and pharmacology of elapid cytotoxins. Pharmacol. Ther. 36, 1-40. 1988.

Edward H. Kerns. In Vivo Studies of Brain Exposure in Drug Discovery, Chapter 13

Endo and Tamiya. Structure-function relationship of postsynaptic neurotoxins from snake venoms. In: Harvey, A. L. (Ed.), Snake Toxins. Pergamon Press, New York, pp. 165-222.1991.

Gao Hongjing. Separation and purification of Neurotoxin from Naja Atra and study of its analgesic effect. Thesis of Fujian Medical University, 2014

Gasanov S E, Alsarraj M A, Gasanov N E, Rael E D. Cobra venom cytotoxin free of phospholipase A2 and its effect on model membranes and T leukemia cells. J Membr Biol 155:133-42. 1997.

Gasanov S E, Dagda R K, Rael E D. Snake venom cytotoxins, phospholipase A2s, and Zn2+-dependent metalloproteinases: mechanisms of action and pharmacological relevance. J Clin Toxicol 4:1000181. 2014

Gabrielsson J, Dolgos H, Gillberg P G, Bredberg U, Benthem B, Duker G. Early integration of pharmacokinetic and dynamic reasoning is essential for optimal development of lead compounds: strategic considerations. Drug Discov Today 14(7-8):358-372. 2009

Hammarlund-Udenaes M. Active-site concentrations of chemicals—are they a better predictor of effect than lasma/organ/tissue concentrations? Basic Clin Pharmacol Toxicol 106(3):215-220. 2009

Joubert, F. J., Taljaard, N, Snake venoms. The amino-acid sequence of protein S2C4 from Dendroaspis jamesoni kaimosae (Jameson's mamba) venom. Hoppe Seylers. Z. Physiol. Chem. 360, 571-580. 1979

Laustsen, A. H. Toxin synergism in snake venoms. Toxin Reviews, 35(3-4), 165-170. DOI: 10.1080/15569543. 2016

Li Di and Edward H. Kerns. Blood-Brain Barrier in Drug Discovery: Optimizing Brain Exposure of CNS Drugs and Minimizing Brain Side Effects for Peripheral Drugs, First Edition. John Wiley & Sons, Inc. 2015

Lin Lili, Xu Yunlu. Laboratory of Venom Research, Fujian Medical University, Fuzhou 360004, China. Strait Pharmaceutical Journal Vol 21 No.5, 2009

Morgan P, Van Der Graaf P H, Arrowsmith J, Feltner D E, Drummond K S, Wegner C D, Street S D. Can the flow of medicines be improved? Fundamental pharmacokinetic and pharmacological principles toward improving Phase II survival. Drug Discov Today 17(9-10):419-424. 2012

Ming zhixue, huanglu. Study of wannan cobra venom analgesic factor, (CVAF). Chinese Journal of Pathophysiology. 01, 2013

Nirthanan and Gwee. Three-finger α-neurotoxins and the nicotinic acetylcholine receptor, forty years on. J. Pharmacol. Sci. 94, 1-17. 2004.

Pawlak, J., Mackessy, S. P., Sixberry, N. M., Stura, E. A., Le Du, M. H., Menez, R., Foo, C. S., Menez, A., Nirthanan, S., Kini, R. M., Denmotoxin, a three-finger toxin from the colubrid snake Boiga dendrophila (Mangrove Catsnake) with bird-specific activity. J. Biol. Chem. 281, 29030-29041, 2008.

Pawlak, J., Mackessy, S. P., Sixberry, N. M., Stura, E. A., Le Du, M. H., Menez, R., Foo, C. S., Menez, A., Nirthanan, S., Kini, R. M. Irditoxin, a novel covalently linked heterodimeric three-finger toxin with high taxonspecific neurotoxicity. FASEB J. 23, 534-545. 2009.

Pawlak, J., Kini, R. M. Unique gene organization of colubrid threefinger toxins: complete cDNA and gene sequences of denmotoxin, a bird-specific toxin from colubrid snake Boiga dendrophila (Mangrove Catsnake). Biochimie. 90, 868-877, 2008.

Pravat Thatoi, Ritesh Acharya, Ashish Malla. Acute respiratory failure following neurotoxic snake bite—A study of 101 cases of neurotoxic snake bite from eastern India. European Respiratory Journal 48: PA2140; 2016

R. Manjunatha Kini, Robin Doley. Structure, function and evolution of three-finger toxins: Mini proteins with multiple targets. Toxicon 56, 855-867. 2010

R. Manjunatha Kini a,b,*, Robin Doley a,c. Structure, function and evolution of three-finger toxins: Mini proteins with multiple targets. Toxicon 56 855-867. 2010

Ruan, K. -H., Spurlino, J., Quiocho, F. A., and Atassi, M. Z. Acetylcholine receptor-alpha-bungarotoxin interactions. Proc. Natl. Acad. Sci. USA Aug;87(16):6156-60. 1990

Ruan, K. -H., Stiles, B. G., and Atassi, M. Z. The short neurotoxin binding regions on the α-chain of human. Biochem. J. 274, 849-854. 1991

Ruzhu Chen, Susan E. Robinson. The effect of cholinergic manipulations on the analgesic response to cobrotoxin in mice. Life Sciences, Volume 47, Issue 21, Pages 1949-1954, 1990

Ruzhu Chen, Susan E. Robinson. THE EFFECT OF COBROTOXIN ON THE TURNOVER OF ACETYLCHOLINE IN MOUSE' BRAIN, Journal of Sun Yat-sen University Medical Sciences, 02-1990

Ruzhu Chen, Susan E. Robinson. The effect of cobrotoxin on cholinergic neurons in the mouse. Life Sci. 51(13):1013-9, 1992

Smith D A, Di L, Kerns E H. The effect of plasma protein binding on in vivo efficacy: misconceptions in drug discovery. Nat Rev Drug Discov 9 (12):929-939. 2010

Liu X, Chen C, Hop C E. Do we need to optimize plasma protein and tissue binding in drug discovery? Curr Top Med Chem 11(4):450-466. 2011

Li Di and Edward H. Kerns. Blood-Brain Barrier in Drug Discovery: Optimizing Brain Exposure of CNS Drugs and Minimizing Brain Side Effects for Peripheral Drugs, First Edition. Published 2015 by John Wiley & Sons, Inc.

Shengwei Xiong, Chunhong Huang. Synergistic strategies of predominant toxins in snake venoms. Toxicology Letters 287, 142-154. 2018

Strydom D J, Botes D P. Snake venom toxins-I. Preliminary studies on the separation of toxins of elapidae venoms. Toxicon 8:203-9. 1970

Vagish Kumar, Laxman Shanbhag. Applications of snake venoms in treatment of cancer. Asian Pacific Journal of Tropical Biomedicine, Volume 5, Issue 4, Pages 275-276. April 2015

Tsetlin V I , Karlsson E , Utkin YuN. Interacting surfaces of neurotoxins and acetylcholine receptor. Toxicon Volume 20, Issue 1, Pages 83-93, 1982.

V. I Tsetlin, FHucho. Snake and snail toxins acting on nicotinic acetylcholine receptors: Fundamental aspects and medical applications FEBS Letters Volume 557, Issues 1-3, Pages 9-13, 16 Jan., 2004

William Kielbasa, Robert E. Stratford, Jr. Microdialysis to Assess Free Drug Concentration in Brain. 1 Eli Lilly and Company, Indianapolis, Ind., USA 2 Xavier University of Louisiana, New Orleans, La., USA. 2015

Zhu Tianxin, Yuan Caijun, Preparation of neurotoxin from cobra venom on a large scale and its analgesic effect. West China Journal of Pharmaceutical Sciences. 22(3): 247˜249, 2007

1. A method for treating pain in a mammal. Said method comprising administering to a mammal in need thereof a pharmaceutical composition of a therapeutically effective amount of Chain A, Cobrotoxin, or Chain A, Cobrotoxin B; and a pharmaceutically acceptable carrier base for use in alleviating or controlling pain.

2. The method of claim 1 where Chain A, Cobrotoxin and Chain A, Cobrotoxin B can be from elapid, sea snake, recombination resource, or from chemical synthesis.

3. The method of claim 1 for parenteral (intravenous, intramuscular, Intraarticular, intrathecal or subcutaneous), nasal, oral, sublingual or rectal administration ranging from 1 μg/Kg to 350 μg/Kg.

4. The method of claim 1 for topical administration comprising between 50 μg and 500 μg per gram of base.

5. The method of claim 1 comprising administering the composition ranging from once per year to several times a day.
